(19) United States
(12) Patent Application Publication
(10) Pub. No.: US 2012/0190758 A1
(43) Pub. Date: Jul. 26, 2012

(54) SUPPORTED COBALT CATALYSTS FOR THE FISCHER TROPSCH SYNTHESIS

(75) Inventors: Peter Trenton BISHOP, Reading (GB); Peter Richard ELLIS, Reading (GB)

(73) Assignee: JOHNSON MATTHEY PLC, London (GB)

(21) Appl. No.: 13/439,210

(22) Filed: Apr. 4, 2012

Related U.S. Application Data
(62) Division of application No. 11/993,542, filed on Dec. 21, 2007.

(30) Foreign Application Priority Data
Jun. 23, 2005 (GB) 0512791.5

(51) Int. Cl. C07C 1/04

(52) U.S. Cl. 518/713; 518/717

(57) ABSTRACT

A process for the synthesis of hydrocarbons by the Fisher Tropsch process includes reacting a mixture of carbon monoxide and hydrogen at elevated temperature and pressure in the presence of a catalyst including 15-50% wt cobalt at least partially in elemental form, supported on an oxidic support of aluminium, oxygen and 0.5-10% wt lithium, where the oxidic support includes lithium oxides and >75% wt of the lithium oxides are lithium aluminate spinel, LiAl₅O₈.

### CROSS-REFERENCE TO RELATED APPLICATIONS

This application is a divisional application of U.S. patent application Ser. No. 11/993,542, filed Dec. 21, 2007, which is the U.S. National Phase of PCT International Application No. PCT/GB2006/050143, filed Jun. 8, 2006, and claims priority of British Patent Application No. 0512791.5, filed Jun. 23, 2005, the disclosures of all of which are incorporated herein by reference in their entireties for all purposes.

### FIELD OF THE INVENTION

This invention relates to supported catalysts and in particular to supported cobalt catalysts suitable for the Fischer-Tropsch synthesis of hydrocarbons.

### BACKGROUND OF THE INVENTION

Cobalt catalysts suitable for the Fischer-Tropsch synthesis of hydrocarbons are known and in their active form typically comprise elemental or zero-valent cobalt supported on an oxidic support such as alumina, silica or titania.

Preparation of supported cobalt catalysts suitable for the Fischer-Tropsch synthesis of hydrocarbons has typically been by impregnation of soluble cobalt compounds into ‘pre-formed’ oxidic support materials or by precipitation of cobalt compounds from solution in the presence of support powders or extrudates, followed by a heating step in air and then, prior to use, activation of the catalyst by reduction of the resulting cobalt compounds in the catalyst precursors to elemental, or ‘zero-valent’ form typically using a hydrogen-containing gas stream. The step of heating in air converts at least some of the cobalt compounds to cobalt oxide, Co₃O₄ and the subsequent reduction with hydrogen converts the Co₃O₄ to cobalt monoxide, CoO, and thence the catalytically active cobalt metal.

However, prolonged heating of the catalyst precursor at high temperature during its manufacture has been found to reduce the resulting cobalt surface area of the subsequently reduced catalysts, possibly as a result of increased support-metal interactions leading to undesired formation of spinel or other complex oxides. For example, heating cobalt compounds on alumina in air can increase cobalt aluminate formation. In the subsequent catalyst activation, cobalt aluminate is more resistant to reduction with hydrogen than cobalt oxide, requiring prolonged reduction times or increased temperatures. Both of these can lead to reduced cobalt surface areas in the resulting catalysts.

### SUMMARY OF THE INVENTION

Whereas silica and titania-supported catalysts may be prepared, alumina-supported catalysts present some advantages over other supported catalysts. For example, alumina-supported catalysts are easier to shape by extrusion than a silica, titania, or zirconia-supported catalysts and the mechanical strength of the resulting catalyst is often higher. Furthermore, in reactions where water is present, silica can be unstable. Alumina is more stable under such conditions.

As cobalt surface area has been found to be proportional to catalyst activity, an alumina support which is resistant to cobalt aluminate formation is desired.

Accordingly, the invention provides a catalyst comprising 5-75% wt cobalt supported on an oxidic support consisting of aluminium and 0.01-20% wt lithium.

The invention further provides a process for preparing the catalyst, comprising (i) preparing an oxidic support by impregnating an alumina with a solution of a lithium compound, drying the impregnated support and heating to convert the lithium compound to one or more lithium oxides, (ii) impregnating the oxidic support with a solution of a cobalt compound or precipitating an insoluble cobalt compound in the presence of the support, and (iii) optionally calcining the resulting composition.

The catalyst precursor thus produced may be converted into its active form for the Fischer-Tropsch reaction by the step of heating the resulting catalyst precursor in the presence of a reducing gas to reduce at least a portion of the cobalt to elemental form.

The invention further provides the use of the cobalt catalyst for the Fischer-Tropsch synthesis of hydrocarbons.

### BRIEF DESCRIPTION OF THE DRAWINGS

The invention is best understood from the following detailed description when read in connection with the accompanying drawing. Included in the drawing are the following figures:

FIG. 1 is a depiction of a Fourier Transform Infrared (FTIR) spectrum of a cobalt oxide coated catalyst precursor prepared using lithium/aluminum oxide according to exemplary embodiments of the invention; and

FIG. 2 is a comparative depiction of a FTIR spectrum of a cobalt oxide coated catalyst precursor prepared using uncoated gamma alumina.

### DETAILED DESCRIPTION OF THE INVENTION

U.S. Pat. No. 6,184,416 describes lithium aluminate as a catalyst support for rhodium-catalysed hydrogenation of aromatic amines. The lithium aluminate conferred increased water tolerance and improved attrition resistance. However U.S. Pat. No. 6,184,416 does not describe cobalt Fischer-Tropsch catalysts nor does it contemplate the problem of cobalt aluminate formation. We have found that for cobalt Fischer-Tropsch catalysts, where cobalt aluminate formation can be a problem that the present invention offers improved cobalt catalyst performance.

The oxidic catalyst support comprises 0.01-20%, preferably 0.5-10%, more preferably 1-5% Li by weight. The lithium to aluminium atomic ratio is preferably 0.08-0.8. The lithium oxide may be in the form of lithia (Li₂O) but preferably comprises lithium aluminate spinel (LiAl₅O₈). More preferably the lithium oxides comprise >75% wt lithium aluminate, particularly >90% wt lithium aluminate. Thus preferably the lithium is predominantly in the form of lithium aluminate. This is believed to confer improved water resistance to the catalyst as well as reduce cobalt aluminate formation.

The oxidic support may be in the form of a powder or of a shaped unit such as a granule, tablet or extrudate. Shaped units may be in the form of elongated cylinders, spheres, lobed or fluted cylinders or irregularly shaped particles, all of which are known in the art of catalyst manufacture. Alternatively the support may be in the form of a coating upon a structure such as a honeycomb support, monolith etc.

A suitable powder catalyst support generally has a surface-weighted mean diameter D[3,2] in the range 1 to 200 μm. In certain applications such as for catalysts intended for use in slurry reactions, it is advantageous to use very fine particles which have a surface-weighted mean diameter D[3,2] in the range 1 to 20 μm, e.g. 1 to 10 μm. For other applications e.g. as a catalyst for reactions carried out in a fluidised bed, it may be desirable to use larger particle sizes, preferably in the range 50 to 150 μm. The term surface-weighted mean diameter D[3,2], otherwise termed the Sauter mean diameter, is defined by M. Alderliesten in the paper “A Nomenclature for Mean Particle Diameters”; Anal. Proc., vol 21, May 1984, pages 167-172, and is calculated from the particle size analysis which may conveniently be effected by laser diffraction for example using a Malvern Mastersizer.

The oxidic support may be prepared by impregnating an alumina with a solution of a lithium compound.

The alumina may be a hydrated alumina such as gibbsite (Al(OH)₃) or boehmite (AlO(OH)) but the alumina is preferably a transition alumina, so that preferred catalysts according to the invention comprise a cobalt species on a lithium aluminate-containing transition alumina support. A suitable transition alumina may be of the gamma-alumina group, for example eta-alumina or chi-alumina. These materials may be formed by calcination of aluminium hydroxides at 400 to 750° C. and generally have a BET surface area in the range 150 to 400 m²/g. Alternatively, the transition alumina may be of the delta-alumina group which includes the high temperature forms such as delta- and theta-aluminas which may be formed by heating a gamma group alumina to a temperature above about 800° C. The delta-group aluminas generally have a BET surface area in the range 50 to 150 m²/g. Alternatively, we have found that suitable catalyst supports may comprise an alpha-alumina. The transition aluminas contain less than 0.5 mole of water per mole of Al₂O₃, the actual amount of water depending on the temperature to which they have been heated.

The pore volume of the alumina is preferably >0.4 cm³/g.

Where the transition alumina is a precipitated alumina, e.g. a precipitated gamma alumina, we have found that improved catalyst performance may be achieved when the precipitated alumina is washed with water and/or acid and/or ammonia solutions to remove soluble contaminants such as alkali metals and/or sulphur and/or chlorine, prior to impregnating the alumina with lithium. In particular we have found that sequentially washing a precipitated alumina with nitric acid and ammonia solutions, followed by a water wash, can remove Na and S and Cl contaminants that otherwise may reduce FT catalyst activity and/or selectivity to C5+ hydrocarbons.

One or more suitably soluble lithium compounds may be used for the impregnation, such as lithium nitrate, lithium oxalate or lithium acetate, preferably lithium nitrate. Water is the preferred solvent. Single or multiple impregnations may be performed to achieve a desired lithium level. The impregnated support may, if desired, be separated from any excess solution before drying to remove solvent. Following drying, the impregnated alumina is heated, preferably in air, to effect a physiochemical change whereby the lithium compound is converted to lithium oxides. Drying is preferably effected at 20-150° C., preferably 90-120° C. for up to 24 hours. Drying may be performed in air or under an inert gas such as nitrogen or argon, or in a vacuum oven. Calcination, preferably in air or possibly another oxygen-containing gas is preferably carried out at temperatures in the range from 500-1500° C., preferably 700-1000° C. to ensure the formation of lithium oxides: Calcination may be performed up to 24 hours preferably <16 hours. Thus the oxidic support may be described as a lithium oxide or lithium aluminate-coated alumina, where the amount of alumina remaining depends upon the amount of lithium present.

If desired, the lithium oxide-containing oxidic support may be washed with water and/or acid/and or ammonia solutions to remove soluble contaminants such as alkali metals and/or sulphur or chlorine, prior to combining the support with cobalt compounds.

Cobalt is combined with the oxidic support to prepare the catalyst. The catalyst contains 5-75% wt cobalt (as atoms). Preferably the catalyst contains 15-50% wt Co, more preferably 5-40% wt cobalt. The cobalt may be in elemental, zero-valent form in which the catalyst is active for the Fischer-Tropsch reactions, or may be in the form of cobalt compounds, such as cobalt oxide, which are precursors to the active catalyst. The precursors are converted to the active catalyst preferably by treatment with a reducing gas prior to use. Thus the term “catalyst” herein relates to active catalyst or catalyst precursor.

The cobalt may be combined with the oxidic support by impregnation using a solution of a suitable cobalt compound or by precipitation of cobalt compounds from solution. Impregnation is particularly suitable for preparing catalysts containing between 5 and 40% by weight cobalt.

Precipitation may be effected by action of a base on acidic cobalt salts such as cobalt nitrate, cobalt acetate or cobalt formate, or by heating a cobalt ammine carbonate solution, for example as described in WO 01/87480 and in particular WO 05/107942. Precipitation may be used to prepare catalysts containing 5-75% wt cobalt, particularly catalysts containing >20% wt cobalt, especially catalysts containing >40% wt cobalt.

Methods for producing cobalt catalysts are well known and generally comprise combining a catalyst support with a solution of cobalt, e.g. cobalt nitrate, cobalt acetate, cobalt formate, cobalt oxalate, or cobalt ammine carbonate at a suitable concentration. An incipient wetness technique may preferably be used whereby sufficient cobalt solution to fill up the pores of the support material added to the catalyst support. Alternatively larger amounts of cobalt solution may be used if desired. Whereas a number of solvents may be used such as water, alcohols, ketones or mixtures of these, preferably the support has been impregnated using aqueous solutions. Impregnation of aqueous cobalt nitrate is preferred. Single or multiple impregnations may be performed to achieve a desired cobalt level in the catalyst precursor. In another preferred embodiment, insoluble cobalt compounds are precipitated onto the oxidic support from an aqueous solution of cobalt ammine carbonate.

If desired, the cobalt-containing support may be dried to remove solvent. The drying step may be performed at 20-120° C., preferably 95-110° C., in air or under an inert gas such as nitrogen, or in a vacuum oven.

The dried Co-containing oxidic support may then be calcined, i.e. heated, preferably in air, or another oxygen-containing gas under oxidising conditions, to convert cobalt compounds impregnated or precipitated onto the lithium oxide-coated alumina into cobalt oxide (CO₃O₄). Alternatively, particularly where the cobalt compound is cobalt formate, heating may be performed under non-oxidising conditions under which at least a portion of the cobalt compound will decompose to form cobalt metal. The heating (calcination) temperature is preferably in the range 130 to 500° C. but the maximum calcination temperature is preferably ≦450° C., more preferably ≦400° C., most preferably ≦350° C., especially ≦300° C. to minimize cobalt-support interactions. The calcination time is preferably ≦24, more preferably ≦16, most preferably ≦8, especially ≦6 hours.

Alternatively, the calcination step may be omitted so that the subsequent reduction step is performed directly on the dried impregnated or precipitated cobalt compounds. Where cobalt nitrate is impregnated onto the oxidic support, preferably a calcination step, is included so that at least some of the cobalt compounds are converted into cobalt oxide. A calcination step is not required where of insoluble cobalt compounds have been precipitated from a solution of cobalt ammine carbonate, as the precipitated compounds may already comprise Co₃O₄.

Where the cobalt is derived from cobalt nitrate, if desired, the calcined cobalt-impregnated support, after cooling, may be heated to a temperature below 250° C., preferably 50-225° C., in the presence of a gas mixture comprising 0.1-10% hydrogen by volume in an inert gas such as nitrogen, to effect further denitrification of the catalyst support. This is particularly useful when calcination of the cobalt catalyst precursor has been performed at ≦400° C., particularly ≦300° C. Under these conditions essentially no reduction of the cobalt oxide takes place.

The drying, calcination and/or subsequent denitrification may be carried out batch-wise or continuously, depending on the availability of process equipment and/or scale of operation.

The catalyst may in addition to cobalt, further comprise one or more suitable additives or promoters useful in Fischer-Tropsch catalysis. For example, the catalysts may comprise one or more additives that alter the physical properties and/or promoters that effect the reducibility or activity or selectivity of the catalysts. Suitable additives are selected from compounds of metals selected from molybdenum (Mo), copper (Cu), iron (Fe), manganese (Mn), titanium (Ti), zirconium (Zr), lanthanum (La), cerium (Ce), chromium (Cr), magnesium (Mg) or zinc (Zn). Suitable promoters include silver (Ag), gold (Au), rhodium (Rh), iridium (Ir), ruthenium (Ru), rhenium (Re), nickel (Ni), platinum (Pt) and palladium (Pd). Preferably one or more promoters selected from Cu, Ag, Au, Ni, Pt, Pd, Ir, Re or Ru are included in the catalyst, more preferably Ni, Pt, Pd, Ir, Re or Ru. Additives and/or promoters may be incorporated into the catalyst via the precursor by use of suitable compounds such as acids, e.g. perrhenic acid, metal salts, e.g. metal nitrates or metal acetates, or suitable metal-organic compounds, such as metal alkoxides or metal acetylacetonates. Typical amounts of promoters are 0.1-10% metal by weight on cobalt. If desired, the compounds of additives and/or promoters may be added in suitable amounts to the cobalt impregnation solutions. Alternatively, they may be combined with the catalyst precursor before or after drying/denitrification.

To render the catalyst catalytically active for Fischer-Tropsch reactions, at least a portion of the cobalt oxide may be reduced to the metal. Reduction is preferably performed using hydrogen-containing gasses at elevated temperature. Preferably >75% of the cobalt is reduced.

Before the reduction step, the catalyst may, if desired, be formed into shaped units suitable for the process for which the catalyst is intended, using methods known to those skilled in the art.

Reduction may be performed by passing a hydrogen-containing gas such as hydrogen, synthesis gas or a mixture of hydrogen with nitrogen or other inert gas over the oxidic composition at elevated temperature, for example by passing the hydrogen-containing gas over the catalyst precursor at temperatures in the range 300-600° C. for between 1 and 16 hours, preferably 1-8 hours. Preferably the reducing gas comprises hydrogen at >25% vol, more preferably >50% vol, most preferably >75%, especially >90% vol hydrogen. Reduction may be performed at ambient pressure or increased pressure, i.e. the pressure of the reducing gas may suitably be from 1-50, preferably 1-20, more preferably 1-10 bar abs. Higher pressures >10 bar abs may be more appropriate where the reduction is performed in-situ.

Catalysts in the reduced state can be difficult to handle as they can react spontaneously with oxygen in air, which can lead to undesirable self-heating and loss of activity. For catalysts suitable for Fischer-Tropsch processes, the reduced catalyst is preferably protected by encapsulation of the reduced catalyst particles with a suitable barrier coating. In the case of a Fischer-Tropsch catalyst, this may suitably be a FT-hydrocarbon wax. Alternatively, the catalyst can be provided in the oxidic unreduced state and reduced in-situ with a hydrogen-containing gas. Whichever route is chosen, the cobalt catalysts prepared from precursors obtained by the method of the present invention provide high metal surface areas per gram of reduced metal. For example, the cobalt catalyst precursors, when reduced by hydrogen at 425° C., preferably have a cobalt surface area of ≧20 m²/g of cobalt as measured by H₂ chemisorption at 150° C. More preferably the cobalt surface area is ≧30 m²/g cobalt and most preferably ≧40 m²/g cobalt. Preferably, in order to achieve a suitable catalyst volume in Fischer-Tropsch processes, the catalysts have a cobalt surface area/g catalyst ≧5 m²/g catalyst, more preferably ≧8 m²/g catalyst.

The cobalt surface area may be determined by H₂ chemisorption. A preferred method is as follows; Approximately 0.2 to 0.5 g of sample material, e.g. catalyst precursor, is firstly degassed and dried by heating to 140° C. at 10° C./min in flowing helium and maintaining at 140° C. for 60 minutes. The degassed and dried sample is then reduced by heating it from 140° C. to 425° C. at a rate of 3° C./min under a 50 ml/min flow of hydrogen and then maintaining the hydrogen flow at 425° C. for 6 hours. Following this reduction, the sample is heated under vacuum to 450° C. at 10° C./min and held under these conditions for 2 hours. The sample is then cooled to 150° C. and maintained for a further 30 minutes under vacuum. The chemisorption analysis is then carried out at 150° C. using pure hydrogen gas. An automatic analysis program is used to measure a full isotherm over the range 100 mm Hg up to 760 mm Hg pressure of hydrogen. The analysis is carried out twice; the first measures the “total” hydrogen uptake (i.e. includes chemisorbed hydrogen and physisorbed hydrogen) and immediately following the first analysis the sample is put under vacuum (<5 mm Hg) for 30 mins. The analysis is then repeated to measure the physisorbed uptake. A linear regression is then applied to the “total” uptake data with extrapolation back to zero pressure to calculate the volume of gas chemisorbed (V).

Cobalt surface areas may then be calculated using the following equation;

Co surface area=(6.023×10²³×V×SF×A)/22414

where
V=uptake of H₂ in ml/g
SF=Stoichiometry factor (assumed 2 for H₂ chemisorption on Co)
A=area occupied by one atom of cobalt (assumed 0.0662 nm²)

This equation is described in the Operators Manual for the Micromeretics ASAP 2010 Chemi System V 2.01, Appendix C, Part No. 201-42808-01, October 1996.

The catalysts may be used for the Fischer-Tropsch synthesis of hydrocarbons.

The Fischer-Tropsch synthesis of hydrocarbons with cobalt catalysts is well established. The Fischer-Tropsch synthesis converts a mixture of carbon monoxide and hydrogen to hydrocarbons. The mixture of carbon monoxide and hydrogen is typically a synthesis gas having a hydrogen:carbon monoxide ratio in the range 1.7-2.5:1. The reaction may be performed in a continuous or batch process using one or more stirred slurry-phase reactors, bubble-column reactors, loop reactors or fluidised bed reactors. The process may be operated at pressures in the range 0.1-10 Mpa and temperatures in the range 150-350° C. The gas-hourly-space velocity (GHSV) for continuous operation is in the range 100-25000 hr⁻¹. The catalysts of the present invention are of particular utility because of their high cobalt surface areas/g catalyst.

### EXAMPLES

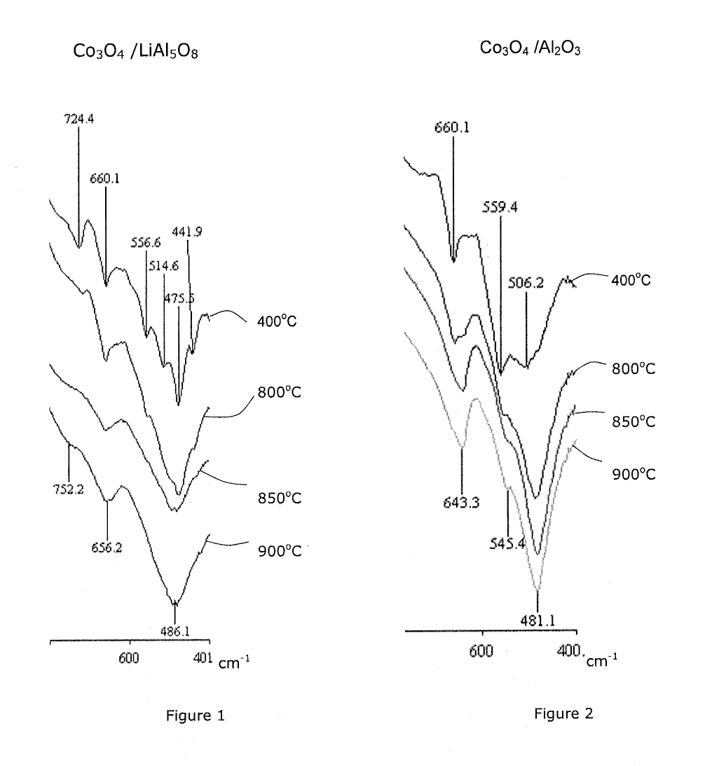

The invention will now be further described by reference to the following Examples and by reference to FIGS. 1 and 2 which depict FTIR spectra of cobalt oxide coated catalyst precursors prepared using lithium/aluminium oxide and uncoated gamma alumina respectively.

### Example 1

### Preparation of Catalyst Support

Lithium nitrate trihydrate (4.18 g, 33.5 mmol Li) was dissolved in 16 ml demineralised water. To this was then added gamma-alumina (grade HP14-150 from Sasol) 15.8 g, and the resulting mixture thoroughly stirred. The damp solid was transferred to a 400 ml beaker and dried at 105° C. for 3½ hours. The dried material was transferred to a ceramic tray and calcined by heating in air to 800° C., holding at 800° C. for four hours then cooling to room temperature. The heating and cooling rates were both 10° C./min. The Li content=2.7% and Li:Al=0.22. X-ray diffractometry (XRD) showed that the Li was essentially all present as lithium aluminate, LiAl₅O₈.

### Example 2

### Preparation of Catalyst

### (a) Impregnation Using Cobalt Nitrate Solution

Cobalt nitrate hexahydrate (18.90 g, 64.9 mmol Co) was dissolved in 8.6 ml demineralised water, giving a red solution. Lithium oxide coated alumina prepared according to the method of example 1 (15.30 g) was added in one portion to the cobalt solution, giving a pink solid on stirring. The damp solid was transferred to a 400 ml beaker and dried at 105° C. for three hours. The dry solid was transferred to a ceramic tray, and calcined by heating in air to 400° C. at 2° C./min, holding at 400° C. for one hour then cooling to room temperature. The product was a black solid. The cobalt content was 18.9% wt and the lithium content 1.07% wt.

To determine the relative stability to the formation of blue cobalt aluminate, small amounts (ca.

1.4 g) of the catalyst precursor and a comparative catalyst precursor prepared using un-modified gamma alumina were heated in air to 800, 850 or 900° C. at 10° C./min, held at temperature for two hours then cooled to room temperature at 10° C./min.

Visual inspection shows the lithium aluminate supported catalyst to retain its dark colour compared to the un-modified gamma-alumina supported catalyst. This indicates that more of the cobalt has remained in the more readily reducible black Co₃O₄ form and has not been converted to blue cobalt aluminate.

Colourimetry data was obtained using a Datacolor International Spectraflash 500 colorimeter. L, a, b, c and h values were recorded for the samples. L=lightness with black 0 & white 100; a=green—red with negative values green and positive values red; b=blue-yellow with negative values blue and positive values yellow, c=colour strength and h=hue angle. The results are given below;

| | Heating T | | | | | |
|---|---|---|---|---|---|---|
| Sample | (° C.) | L | a | b | c | h |
| Co on | 800 | 13.56 | −5.10 | −15.79 | 16.59 | 251.12 |
| unmodified | 850 | 21.46 | 2.90 | −38.59 | 38.70 | 274.30 |
| alumina | 900 | 30.96 | 8.54 | −41.00 | 41.88 | 281.77 |
| Example 2(a) | 800 | 11.84 | −2.81 | −0.94 | 2.96 | 198.50 |
| | 850 | 14.06 | −4.94 | −9.48 | 10.69 | 242.48 |
| | 900 | 17.75 | −7.67 | −16.93 | 18.56 | 245.64 |

The colourimetry confirms that the catalyst precursor according to the present invention is less prone to form blue cobalt aluminate, than the unmodified material.

The FTIR spectra of the catalyst precursor samples between 400-800 cm⁻¹ are depicted in FIGS. 1 (Co₃O₄/LiAl₅O₈ according to the invention) and 2 (Co₃O₄/Al₂O₃ not according to the invention). The FTIR spectra show a marked difference between the samples, particularly following calcination at 400° C.

A portion of the catalyst precursor prepared according to the invention was transferred to a glass tube, heated to 140° C. at 10° C./minute in flowing helium and held at 140° C. for one hour. The gas flow was changed to hydrogen and the temperature increased to 425° C. at 3° C./minute to affect reduction of the cobalt to elemental form. The temperature was maintained at 425° C. for six hours. The cobalt surface area measured by hydrogen chemisorption at 150° C. following reduction at 425° C. was 8.8 m²/g reduced catalyst, corresponding to 46.6 m²/g cobalt.

### (b) Precipitation from Cobalt Ammine Carbonate Solution

A cobalt hexammine solution with a cobalt content of ˜2.9 w/w % was prepared by the following method. Ammonium carbonate chip (198 g, 30-34 ^{w}/_{w}% NH₃), was weighed into a 5 litre round bottomed flask. Demineralised water (1877 ml) and ammonia solution (1918 ml, Sp.Gr. 0.89) were then added and the mixture stirred until all the ammonium carbonate chip had dissolved. Cobalt basic carbonate (218 g, 45-47 ^{w}/_{w}% Co), was added, with continual stirring, in approximately 25 g aliquots and allowed to dissolve. The final solution was stirred for a minimum of 1 hour to ensure all the cobalt basic carbonate had dissolved. The resulting cobalt hexammine solution was oxidised by the dropwise addition of 67 mls hydrogen peroxide solution (30% concentration) to the stirred solution. During the oxidation process the ORP (Oxidation/reduction potential) increased from −304 mV to −89 mV. Stirring was continued for a further 10 minutes after completion of the peroxide addition by which time the ORP value had dropped to −119 mV. The solution was then filtered.

1960 ml of the cobalt hexammine solution was transferred into a round-bottomed flask situated in an isomantle. The solution was continuously stirred and 42.63 g of the lithium containing gamma alumina support prepared according to the method of Example 1, but having a Li content of 1.40% wt, gradually added (Support:Cobalt ratio=0.75). The system was closed and heat applied. Distillation of the ammonia began as the temperature increased beyond 65° C. The temperature and pH were monitored throughout the preparation. When a pH of 7.5 was reached deposition of the cobalt was deemed to be complete and the preparation ended. The catalyst was immediately filtered then washed with approximately 2 litres of demineralised water. The filter cake was finally dried at 105° C. overnight. The cobalt content of the dried catalyst precursor was 40.5% wt.

The above experiment was repeated using larger amounts of the lithium-containing gamma alumina to obtain catalyst precursors having 29.5% and 20.0% wt Co. The cobalt contents were determined using ICP AES and the cobalt surface areas (CoSA) and % weight loss on reduction (WLOR) determined using hydrogen chemisorption at 150° C. on the precursors reduced at 425° C. according to the method given above. The results are given below;

| | Cobalt content | Co SA | | Co SA |
|---|---|---|---|---|
| Sample | % wt | m² · g⁻¹ cat | % WLOR | m² · g⁻¹ cobalt |
| 2(b)(i) | 20.0 | 21.0 | 15 | 89.3 |
| 2(b)(ii) | 29.5 | 29.5 | 20 | 80.0 |
| 2(b)(iii) | 40.5 | 31.2 | 28 | 55.5 |

Temperature-programmed reduction (TPR) profiles were obtained for the catalysts. Samples of the catalysts were heated between 100 and 1000° C. under a hydrogen-containing gas stream at a set rate and the thermal conductivity difference of the gas stream converted to a profile indicating the consumption of hydrogen coinciding with reduction of Co₃O₄ to CoO and then CoO to Co metal. Compared to comparable catalysts prepared using un-modified alumina, there is a distinct change both in shape and temperature maximum of the CoO to Co metal peak (Tmax 550° C. compared to 650° C.) indicating improved reducibility of the catalysts of the present invention.

Colourimetry data was obtained on the heated precursor containing 20% Co and on a comparative catalyst precursor containing 20% Co, prepared using the same cobalt ammine carbonate method on un-modified alumina. The results are given below;

| | Heating T | | | | | |
|---|---|---|---|---|---|---|
| Sample | (° C.) | L | a | b | c | h |
| Co on | 800 | 16.62 | −3.27 | −23.38 | 23.60 | 262.04 |
| unmodified | 850 | 22.37 | 4.69 | −41.30 | 41.56 | 276.48 |
| alumina | 900 | 22.52 | 4.88 | −41.58 | 41.56 | 276.48 |
| Example 2(b) (i) | 800 | 5.66 | −2.34 | −0.78 | 2.47 | 198.54 |
| | 850 | 9.41 | −7.80 | −6.50 | 10.16 | 219.79 |
| | 900 | 15.05 | −12.59 | −16.62 | 20.86 | 232.85 |

The colourimetry confirms again that the catalyst precursor according to the present invention is less prone to form blue cobalt aluminate, than the unmodified material.

### Example 3

### Catalyst Testing

The cobalt catalyst of Example 2(b) (iii) was used for the Fischer-Tropsch synthesis of hydrocarbons in a laboratory-scale reactor. About 0.1 g of unreduced catalyst mixed with SiC was placed in bed (ca. 4 mm ID by 50 mm depth) and reduced at 430° C. for 420 min in a hydrogen flow of 30 ml/minute. Then hydrogen and carbon monoxide at a 2:1 molar ratio were passed through the bed at 210° C./20 barg. The space velocity was adjusted after 30 hrs to obtain as close as possible 50% CO conversion. The activity and selectivity of the catalyst to CH₄, C2-C4 and C5+ hydrocarbons were measured using known Gas Chromatography (GC) techniques.

A comparative experiment (Comp. 1) was performed under the same conditions using a standard catalyst comprising, prior to reduction, 20% wt Co and 1% wt Re impregnated on an alumina support. The standard catalyst was prepared by impregnating a gamma alumina (Puralox HP14/150) with a solution of cobalt nitrate and ammonium perrhenate, and oven drying the solid at 110° C. for 6.5 hrs before calcination at 200° C. for 1 hour. The catalyst was added at 0.1 g in SIC.

A further comparative experiment was performed under the same conditions using a catalyst prepared by the cobalt ammine carbonate method on an unmodified alumina having a cobalt content of 40% Co (Comp 2).

By noting the relative catalyst composition and space velocity required to give the desired conversion it is possible to calculate a relative activity for the catalyst of the present invention. The results are as follows;

| | Relative | CO₂ | CH₄ | C2-C4 | C5+ | C5=/C5 |
|---|---|---|---|---|---|---|
| Example | Activity | (%) | (%) | (%) | (%) | (%) |
| Comp. 1 | 1.00 | 0.60 | 7.50 | 4.0 | 87.80 | 0.78 |
| Comp. 2 | 2.12 | 1.37 | 8.90 | 7.78 | 81.95 | 0.47 |
| 2(b)(iii) | 2.19 | 0.19 | 8.35 | 4.68 | 86.80 | 0.34 |

The results suggest high activity and especially C5+ hydrocarbon selectivity for the catalysts of the present invention.

What is claimed:

1. A process for the synthesis of hydrocarbons by the Fisher Tropsch process comprising reacting a mixture of carbon monoxide and hydrogen at elevated temperature and pressure in the presence of a catalyst comprising 15-50% wt cobalt at least partially in elemental form, supported on an oxidic support consisting of aluminium, oxygen and 0.5-10% wt lithium, wherein the oxidic support comprises lithium oxides and >75% wt of the lithium oxides are lithium aluminate spinel, LiAl₅O₈.

2. A process according to claim 1 wherein the oxidic support is a powder with a surface-weighted mean diameter in the range 1 to 200 μm.

3. A process according to claim 2 wherein the oxidic support has a surface-weighted mean diameter in the range 1 to 20 μm.

4. A process according to claim 2 wherein the oxidic support has a surface-weighted mean diameter in the range 50 to 150 μm.

5. A process according to claim 1 wherein the oxidic support is in the form of a shaped unit.

6. A process according to claim 1 wherein the Li content of the oxidic support is in the range 1-5% by weight.

7. A process according to claim 1 wherein the catalyst includes one or more promoters selected from the group consisting of Cu, Ag, Au, Ni, Pt, Pd, Ir, Re and Ru.

8. A process according to claim 1 wherein the catalyst, when reduced by hydrogen at 425° C., has a cobalt surface area of ≧20 m²/g of cobalt as measured by H₂ chemisorption at 150° C.

9. A process according to claim 1 wherein yhe mixture of carbon monoxide and hydrogen is a synthesis gas having a hydrogen:carbon monoxide ratio in the range 1.7-2.5:1.

10. A process according to claim 1 wherein the reaction is performed in a continuous or batch process using one or more stirred slurry-phase reactors, bubble-column reactors, loop reactors or fluidised bed reactors.

11. A process according to claim 1 wherein the process is operated at a pressure in the range 0.1-10 MPa

12. A process according to claim 1 wherein the process is operated at a temperature in the range 150-350° C.

13. A process according to claim 1 operated at a gas-hourly-space velocity (GHSV) in the range 100-25000 hr⁻¹.
